(19) United States
(12) Patent Application Publication
(10) Pub. No.: US 2024/0350106 A1
(43) Pub. Date: Oct. 24, 2024

(54) SYSTEMS, METHODS, AND COMPUTER READABLE MEDIA FOR PARAMETRIC FDG PET QUANTIFICATION, SEGMENTATION AND CLASSIFICATION OF ABNORMALITIES

(71) Applicant: University of Virginia Patent Foundation, Charlottesville, VA (US)

(72) Inventors: Bijoy Kundu, Charlottesville, VA (US); Yinlin Li, Charlottesville, VA (US); Carlos Leiva-Salinas, Charlottesville, VA (US); Robert S. Shetlick, Charlottesville, VA (US)

(21) Appl. No.: 18/684,813

(22) PCT Filed: Aug. 17, 2022
(86) PCT No.: PCT/US2022/040621

§ 371 Date: Feb. 19, 2024

Related U.S. Application Data
(60) Provisional application No. 63/233,919, filed on Aug. 17, 2021.

(51) Int. Cl. A61B 6/00; A61B 6/03; A61B 6/50; G01R 33/48; G06T 7/00; G06T 7/11; G06T 7/30; G06V 10/764; G06V 10/766

(52) U.S. Cl. CPC A61B 6/5217; A61B 6/037; A61B 6/507; A61B 6/5247; G01R 33/481; G06T 7/0012; G06T 7/11; G06T 7/30; G06V 10/764; G06V 10/766; G06T 2207/10088; G06T 2207/10104; G06T 2207/20064; G06T 2207/20081; G06T 2207/30096; G06V 2201/03

(57) ABSTRACT

Methods, systems, and computer readable media for performing FDG positron emission tomography (PET) quantification, segmentation, and classification of abnormalities are disclosed. One method includes receiving a plurality of magnetic resonance (MR) images corresponding to a target site of a subject and generating three dimensional (3D) area masks of abnormality volumes from the plurality of MR images. The method further includes segmenting the 3D area masks into one or more individual seed images for each of the abnormality volumes and overlaying the one or more individual seed images onto co-registered parametric PET maps to generate kinetic rate parameters for each of the abnormality volumes. The method also includes utilizing the kinetic rate parameters to train a logistic regression engine to predict a target site condition assessment based on a classification of the abnormality volumes.

### RELATED APPLICATIONS

The presently disclosed subject matter claims the benefit of U.S. Provisional Patent Application Ser. No. 63/233,919, filed Aug. 17, 2021; the disclosure of which is incorporated herein by reference in its entirety.

### TECHNICAL FIELD

The subject matter described herein relates to neurologic disorder analysis and classification methods and fluorodeoxyglucose (FDG) Positron emission tomography (PET) procedures. More particularly, the subject matter described herein relates to methods, systems, and computer readable media for quantification, segmentation, and classification of primary brain tumors in human glioblastoma multiforme (GBM).

### BACKGROUND

Surgical resection, and adjuvant radiotherapy and chemotherapy are the mainstays of disease management in human glioblastoma multiforme (GBM), but there are no curative therapies. Confusingly, brain tissue changes induced by chemoradiotherapy commonly produce a similar MRI appearance, however with markedly different prognostic and therapeutic implications, creating a diagnostic dilemma in the management of GBM patients. Distinguishing tumor progression (TP) vs tumor necrosis (TN) is critical for clinical management decisions. Unfortunately, current magnetic resonance imaging (MRI) techniques have yielded inconsistent results for differentiating between these entities. Positron emission tomography (PET) with Fluorine-18 fluorodeoxyglucose (18F-FDG) serving as a surrogate marker for glucose metabolism, represents an imaging technique that can provide pathophysiologic and diagnostic data in this clinical setting. The current standard of care regarding clinical FDG PET is qualitative visual analysis by performing comparisons between pathologic and normal appearing brain regions with static imaging.

### SUMMARY

A method for performing FDG positron emission tomography (PET) quantification, segmentation, and classification of abnormalities includes receiving a plurality of magnetic resonance (MR) images corresponding to a target site of a subject and generating three dimensional (3D) area masks of abnormality volumes from the plurality of MR images. The method also includes segmenting the 3D area masks into one or more individual seed images for each of the abnormality volumes and overlaying the one or more individual seed images onto co-registered parametric PET maps to generate kinetic rate parameters for each of the abnormality volumes. The method further includes utilizing the kinetic rate parameters to train a logistic regression engine to predict a target site condition assessment based on a classification of the abnormality volumes.

According to another aspect of the method described herein, the MR images are T1-weighted.

According to another aspect of the method described herein, overlaying the 3D area masks onto co-registered parametric PET maps generates a total blood volume (TBV) parameter for each of the abnormality volumes.

According to another aspect of the method described herein, the target site condition assessment includes a tumor progression (TPR) assessment or a treatment related necrosis (TRN) assessment.

According to another aspect of the method described herein, one or more wavelet transforms are utilized to determine the kinetic rate parameters.

According to another aspect of the method described herein, the logistic regression engine is subjected to supervised machine learning (ML) to classify the abnormality volumes.

According to another aspect of the method described herein, receiving the co-registered parametric PET maps corresponding to the target site of the subject.

A system for performing FDG positron emission tomography (PET) quantification, segmentation, and classification of abnormalities, the system includes a PET scanner device configured for configured for collecting volumetric radioactive measurement data associated with an administered radioactive tracer present in a target site of a subject over multiple scanning intervals and generating associated parametric PET maps of the target site and a magnetic resonance (MR) imaging scanner device configured for capturing a magnetic resonance image of the target site. The system further includes a dynamic PET platform including at least one processor, a memory element, and a PET processing engine stored in the memory element and when executed by the at least one processor is configured for receiving a plurality of MR images corresponding to a target site of a subject. The PET processing engine is also configured for generating three dimensional (3D) area masks of abnormality volumes from the plurality of MR images and segmenting the 3D area masks into one or more individual seed images for each of the abnormality volumes. The PET processing engine is further configure for overlaying the one or more individual seed images onto co-registered parametric PET maps to generate kinetic rate parameters for each of the abnormality volumes and utilizing the kinetic rate parameters to train a logistic regression engine to predict a target site condition assessment based on a classification of the abnormality volumes.

According to another aspect of the system described herein, the MR images are T1-weighted.

According to another aspect of the system described herein, the PET processing engine is configured to overlay the 3D area masks onto co-registered parametric PET maps to generate a total blood volume (TBV) parameter for each of the abnormality volumes.

According to another aspect of the system described herein, the target site condition assessment includes a tumor progression (TPR) assessment or a treatment related necrosis (TRN) assessment.

According to another aspect of the system described herein, one or more wavelet transforms are utilized to determine the kinetic rate parameters.

According to another aspect of the system described herein, the logistic regression engine is subjected to supervised machine learning (ML) to classify the abnormality volumes.

According to another aspect of the system described herein, the PET processing engine is configured to receive the co-registered parametric PET maps corresponding to the target site of the subject.

The subject matter described herein may be implemented in hardware, software, firmware, or any combination thereof. As such, the terms “function” “node” or “module” as used herein refer to hardware, which may also include software and/or firmware components, for implementing the feature being described. In one exemplary implementation, the subject matter described herein may be implemented using a computer readable medium having stored thereon computer executable instructions that when executed by the processor of a computer control the computer to perform steps. Exemplary computer readable media suitable for implementing the subject matter described herein include non-transitory computer-readable media, such as disk memory devices, chip memory devices, programmable logic devices, and application specific integrated circuits. In addition, a computer readable medium that implements the subject matter described herein may be located on a single device or computing platform or may be distributed across multiple devices or computing platforms.

### BRIEF DESCRIPTION OF THE DRAWINGS

The subject matter described herein will now be explained with reference to the accompanying drawings of which:

FIG. 1 illustrates a plurality of PET-MR images and associated wavelet transform analysis according to an embodiment of the subject matter described herein;

FIG. 2 is a block diagram of an example computing platform configured for performing dynamic positron emission tomography according to an embodiment of the subject matter described herein;

FIG. 3 is a diagram illustrating the steps for motion correction according to an embodiment of the subject matter described herein;

FIG. 4 is a diagram illustrating the steps for performing dynamic PET MR co-registration according to an embodiment of the subject matter described herein;

FIG. 5 is a diagram illustrating the steps for generating an image-derived blood input function according to an embodiment of the subject matter described herein;

FIG. 6 is a graph of a model corrected blood input function according to an embodiment of the subject matter described herein;

FIG. 7 illustrates examples of area masking using a segmentation engine according to an embodiment of the subject matter described herein;

FIG. 8 illustrates examples of area masks generated from MR images overlaid onto parametric PET maps according to an embodiment of the subject matter described herein;

FIG. 9 depicts a data table containing an example trading data set according to an embodiment of the subject matter described herein;

FIG. 10 depicts a data table containing an example validation and testing data set according to an embodiment of the subject matter described herein;

FIG. 11 depicts a set of dynamic PET scans and associated T1-weighted contrast enhanced MR images according to an embodiment of the subject matter described herein;

FIG. 12 depicts a set of weighted and enhanced MR images according to an embodiment of the subject matter described herein;

FIG. 13 depicts PET imaging and associated plots of recurrent brain tumors according to an embodiment of the subject matter described herein;

FIG. 14 depicts graphs associated with wavelet transform and kinetic analysis according to an embodiment of the subject matter described herein;

FIG. 15 depicts an MRI image and associated graphs of wavelet transform versus standardized uptake values analysis according to an embodiment of the subject matter described herein;

FIG. 16 depicts MR images and dynamic PET images associated with wavelet transform and kinetic analysis according to an embodiment of the subject matter described herein;

FIG. 17 depicts bar graphs associated with the rate of FDG update and wavelet transform analysis according to an embodiment of the subject matter described herein;

FIG. 18 depicts a representative dynamic PET image illustrating the FDG concentration of an example tumor and grey matter according to an embodiment of the subject matter described herein;

FIG. 19 illustrates bar graphs depicting rates of FDG uptake according to an embodiment of the subject matter described herein; and

FIG. 20 is a flow chart illustrating an exemplary process for quantification, segmentation, and classification of abnormalities according to an embodiment of the subject matter described herein.

### DETAILED DESCRIPTION

The disclosed subject matter includes methods, systems, and computer readable media for quantification, segmentation, and classification of abnormalities (e.g., primary brain tumors) in human glioblastoma multiforme (GBM). In particular, the disclosed subject matter pertains to novel methods using a model corrected blood input function accounting for partial volume averaging and peak fitting cost function to compute parametric maps that reveal more information by harnessing kinetic data. In some embodiments, a 4-parameter 3-compartment model from dynamic FDG PET images obtained utilizing the time of flight (TOF) Siemens Biograph PET scanner is used. To highlight the usefulness of these novel mappings, a preliminary prediction algorithm was created using logistic regression that utilizes averaged tumor information from these maps. Preliminary results of tumor segmentation and classification using logistic regression on high resolution parametric PET data are promising in the differentiation of TN from TP in GBM patients based on relevant connections between certain kinetic parameters and the binary prediction outputs.

Notably, GBM accounts for 52 percent of all primary malignant brain tumors, and it is an aggressive cancer with an incidence of 3 per 100,000 adults per year and a median survival of 15 months. Surgery along with adjuvant radiotherapy and chemotherapy remains the mainstay of disease management. However, even with treatment, some tumors recur, appearing on conventional MRI as new enhanced lesions or T2/FLAIR signal abnormalities. Confusingly, post-therapy tissue changes induced by radiotherapy commonly produce a similar MRI appearance (termed “pseudoprogression”), however with markedly different prognostic and therapeutic implications. Indeed, differentiation between active GBM and post-treatment changes such as radiation necrosis is unreliable with MRI and computed tomography (CT) imaging. Distinguishing tumor recurrence from therapy effect is critical for clinical management decisions. Positron emission tomography (PET) with fluorodeoxyglucose (FDG) serving as a marker for glucose metabolism, represents an imaging technique that can provide pathophysiologic and diagnostic data in a clinical setting. The current standard of care regarding clinical FDG PET is qualitative visual analysis by performing comparisons to the contralateral and other brain regions. Standardized uptake values (SUV) measured at a specific time point post-FDG injection have been widely used as a semi-quantitative measure. However, SUV analysis does not reliably differentiate tumor from therapy effect in the standard static imaging protocol. The disclosed subject matter pertains to novel methods that improve the above differentiation by exploiting the diagnostic power of full dynamic/kinetic analysis of the uptake and washout data.

Using the TOF high sensitivity research PET scanner, the disclosed subject matter can be used to perform FDG PET imaging on a plurality of subjects (e.g., 7 subjects). There is compelling evidence that dynamic imaging and wavelet transform (WT) analysis is able to differentiate tumor recurrence from radiation effect. As an example, a subject was followed in two separate dynamic FDG PET scans and resulting data was used to successfully predict and differentiate the above two entities. The first MR scan was performed on this subject 4 weeks post radiation therapy (RT). This was followed by 6 weeks of RT along with an investigator PARP inhibitor during the first 4 weeks. MR images raised the question of tumor recurrence vs radiation effect and hence dynamic FDG PET scan was recommended by the physician in charge.

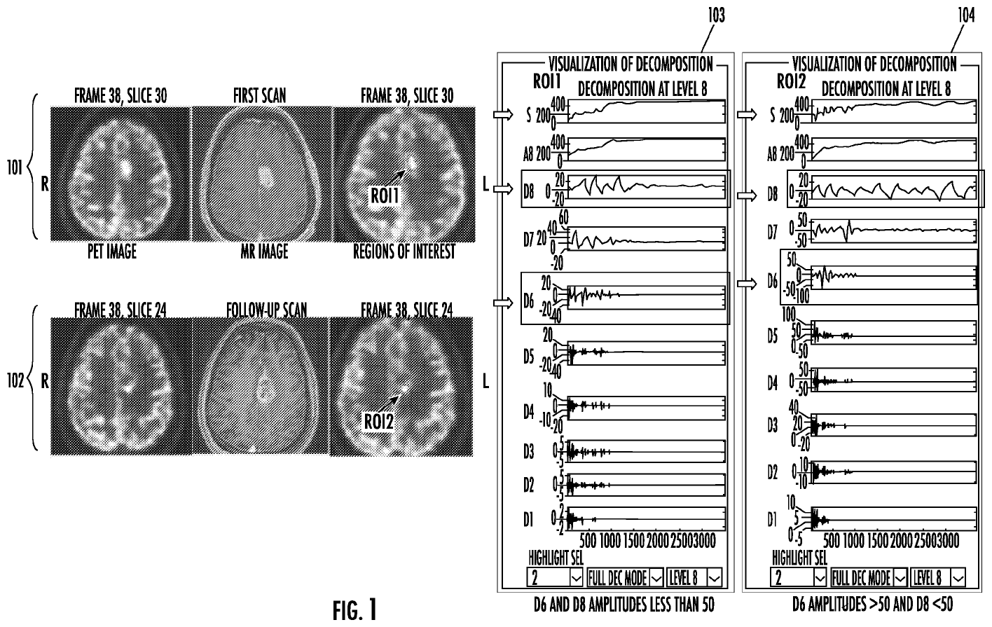

As shown in FIG. 1, the first FDG PET scan 101 indicated enhanced FDG uptake in the PET image (i.e., last five minutes of data shown in FIG. 1; also referred to as a static image 55 minutes post FDG injection) and enhanced contrast in the MR image, indicating tumor recurrence. However, Wavelet Transform (WT) analysis (see ROI 1 in plot 103 in FIG. 1; d6 and d8 members of the wavelet decomposition) of the time-resolved FDG PET data, obtained from the 60 minute dynamic scan, especially at the early time points, indicated radiation effect in FIG. 1. A follow up scan image 102 on the same patient indeed showed that a significant portion of the tumor showed no FDG uptake and lower contrast in the MR scan, which confirmed our prediction. WT analysis of the residual tumor (ROI 2 in plot 104 in FIG. 1) further predicts radiation effect, which was confirmed by 3-6 month interval multidisciplinary clinical evaluation.

In particular, FIG. 1 illustrates PET-MR images (scans 101-102) and wavelet transform (WT) analysis (plots 103-104). The first FDG PET scan post treatment with PARP inhibitor along with radiation indicated active tumor visually in both static PET and MR images (see scan 101).

However, WT analysis (ROI 1) predicted radiation effect (plot 103, ROI 1), which agreed with the follow up scan (image 102). WT analysis on the residual tumor (ROI 2) further predicts radiation effect (plot 104, ROI 2), confirmed by 3-6 month interval multidisciplinary clinical evaluation. In plots 103-104, “s” represents the dynamic PET signal and d6 and d8 represent wavelet decompositions. Further, tumor recurrence criteria for plots 103-104 include a d6 member: a) positive peak value>=50; b) number of peaks>=2 and c) no peak after 4 minutes; d8 member is characterized by a positive peak value of >=50.

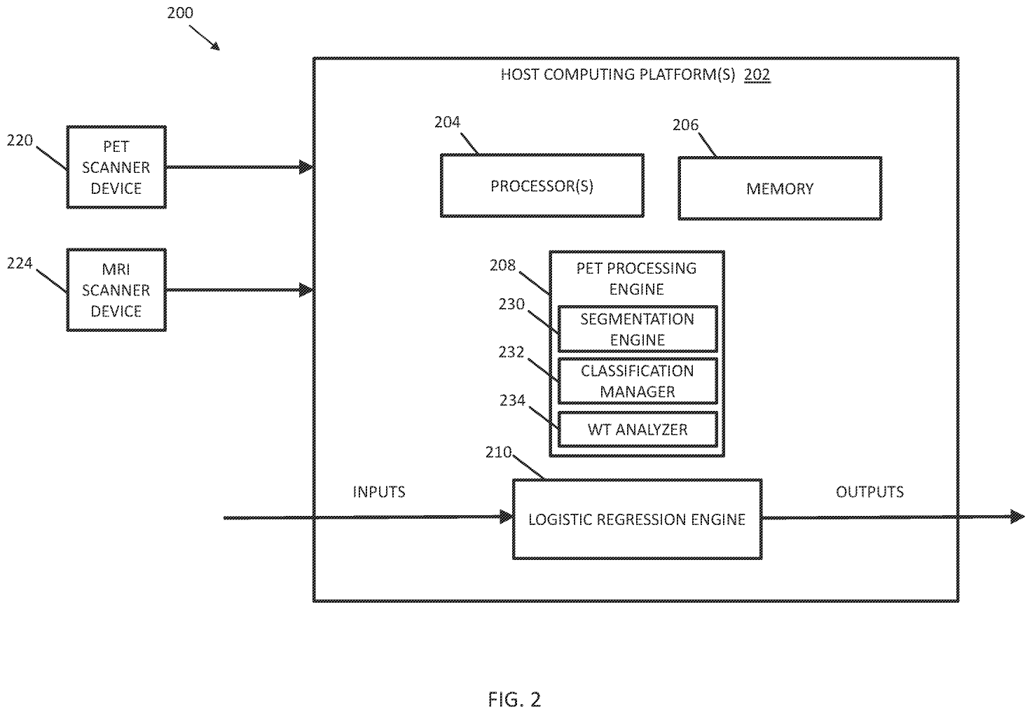

In some embodiment, the PET processing tasks can be conducted using one or more host computing devices. For example, FIG. 2 is a block diagram of an example computer platform system 200 for performing PET quantification, segmentation, classification and associated processes. It will be appreciated that FIG. 2 is for illustrative purposes and that various entities, their locations, and/or their functions may be changed, altered, added, or removed. For example, some entities and/or functions may be combined into a single entity. In another example, an entity and/or function may be located at or implemented by two or more entities.

In FIG. 2, system 200 may include one or more computing platform(s) 202 (e.g., a FDG PET processing platform) having one or more processor(s) 204, such as a central processing unit (e.g., a single core or multiple processing cores), a microprocessor, a microcontroller, a network processor, an application-specific integrated circuit (ASIC), or the like. Computing platform 202 may also include memory 206. Memory 206 may comprise random access memory (RAM), flash memory, a magnetic disk storage drive, and the like. In some embodiments, memory 206 may be configured to store a PET processing engine 208 and a logistic regression engine 210 (e.g., an artificial neural network). PET processing engine 208 may include one or more algorithms, software programs, software processes, and the like. As described below, PET processing engine 208 is configured to control, manage, and administer a plurality of processes corresponding to the execution of the disclosed FDG PET methodology and functionality. In some embodiments, PET processing engine 208 includes a segmentation engine 230, a classification manager 232, and a wavelet transform (WT) analyzer 234. The segmentation engine 230 is responsible for segmenting 3D area masks into individual seed images and the classification manager is configured to predict target site condition assessments (e.g., classifying clinical outcomes between TN vs. TP) as discussed in further detail below. The WT analyzer 234 is responsible for conducting the signaling processing duties associated with the wavelet transform analysis described herein.

In some embodiments, PET processing engine 208 may be configured to receive image data from each of a PET scanner device 220 and/or a MRI scanner device 222. For example, PET scanner device 220 may include a Siemens Biograph time of flight (TOF) mCT scanner that can be utilized to perform dynamic acquisitions of a target site/organ. Further, MRI scanner device 222 may include a Siemens 3T scanner that is configured to captures a high resolution post-contrast T1-weighted MPRAGE MR images (256 pixels×256 pixels×192 slices).

Likewise, logistic regression engine 210 may reside on memory of computing platform(s) 202 and be executable by processor(s) 204. Logistic regression engine 210 may be configured to execute an semi-automated segmentation method (e.g., segment out internal carotid arteries, brain tumors, or other abnormality volumes from PET data).

Notably, as an alternative to conducting classification predictions using wavelet transforms as discussed above with regard to FIG. 1, the disclosed subject matter can be configured to utilize a logistic regression engine configured to predict clinical outcomes (e.g., target site condition assessments). As indicated above, the distinguishing of clinical outcomes, such as tumor progression (TPR) versus treatment related necrosis (TRN), is critical for clinical management decisions in patients with glioblastoma (GBM). Dynamic FDG (dFDG) PET, an advance from traditional static FDG PET, may prove advantageous in clinical staging. Namely, dFDG PET includes novel methods of a model corrected blood input function that accounts for partial volume averaging and includes a peak fitting cost function to compute parametric maps that reveal kinetic rate parameter information (and/or kinetic rate constants). For example, the disclosed subject matter utilizes a 4-parameter 3-compartment model on dFDG PET data. To highlight the usefulness of these novel mappings, a preliminary prediction algorithm was created using various regression methods that utilizes averaged tumor information from these maps. The goal of using dFDG PET and prediction maps in this manner is to better differentiate between TRN and TPR in GBM patients.

As indicated above, Glioblastoma (GBM) is a highly aggressive brain neoplasm with a median survival of 15 months. Surgical resection and adjuvant radiotherapy and chemotherapy are palliative rather than curative. One barrier to treatment is that brain tissue changes induced by chemoradiotherapy commonly produce similar neuroimaging changes to tumor recurrence. As such, distinguishing tumor progression (TPR) from treatment related necrosis (TRN) is critical for clinical management decisions.

Advanced MRI techniques including diffusion and perfusion imaging have yielded inconsistent and unreliable results for differentiating between these entities. PET with fluorine-18 fluorodeoxyglucose (18F-FDG), because it serves as a surrogate marker for glucose metabolism, has been evaluated to help differentiate TRN from TPR. The current standard of care regarding clinical FDG PET is qualitative visual analysis by performing comparisons between pathologic and normal appearing brain regions. Standardized uptake values (SUV, static PET) measured at a specific time point post FDG injection have been widely used as a semi-quantitative measure, but do not reliably differentiate a tumor from post-therapy changes in the standard static PET imaging protocol.

In some embodiments, dFDG PET includes novel methods of a model corrected blood input function that accounts for partial volume averaging and includes a peak fitting cost function to compute parametric maps that reveal kinetic information. The goal of dFDG PET and prediction maps based on dynamic metabolic changes is to improve distinction between TRN and TPR in GBM patients.

### Motion Correction and Image Registration

A dynamic FDG PET scan of the brain can be performed on patient subjects using the Siemens Biograph time of flight (TOF) mCT scanner to produce a DICOM file that contains a complete four dimensional image of each subject's brain tracer update over time. Dynamic acquisition includes an intravenous ˜10 mCi tracer injection over 10 seconds with initiation of a 60-minute scan in list-mode format. PET may be preceded by a high resolution post-contrast T1-weighted MPRAGE MRI (256 pixels×256 pixels×192 slices) using a Siemens 3T scanner for co-registration. Using the combination of each patient subject's T1-weighted MR image and PET image, every potential necrosis region and tumor progression area can be identified within scan. These areas can appear similar at first glance in just the T1-weight image, so each area can be referred to as an “abnormality” or “abnormality volume” and may be assigned a number to distinguish among them. This collection of all abnormalities within one image is what is classified as one “subject” or “patient.”

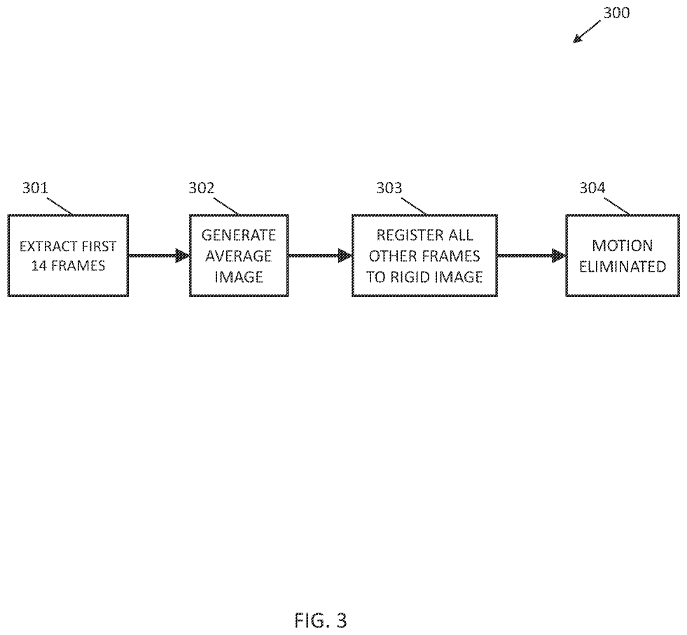

Next, surgical pathology data can be reviewed in combination with expert clinician analysis to conservatively assign the proper clinical outcome label (e.g., TRN or TPR) to all abnormalities within each subject. In cases where there was a combination of TPR and TRN within one abnormality region, the entire region may be labeled as TPR for consistency. For example, there may be between one and five abnormalities per subject. After the labeling analysis concludes, the total number of labeled abnormalities may be n=35 areas, coming from N=26 scanned subjects. IN some embodiments, subsequent processing for each patient subject can be performed with custom tools developed in Matlab (Mathworks Inc., Natick, MA). Image pre-processing may start with motion correction for the 60-minute acquisition to align and lock the anatomy in the same 3 dimensional space throughout the entire time period. PET data (400 pixels×400 pixels×111 slices×38-time frames) can be averaged across the first 14 extracted time frames to create a reference for the proper alignment in the image space (see blocks 301-302 in FIG. 3). This reference may be used to perform a rigid body transform across the 38 frames (see block 303 in FIG. 3). Notably, blocks 301-304 of motion correction process 300 shown in FIG. 3 illustrate exemplary steps for motion correction conducted by a PET processing engine (e.g., PET processing engine 208 in FIG. 2). In some embodiments, motion correction of dynamic PET data can be performed by averaging the first 14 time frames (block 301) to create an average reference image (block 302), which was then used to perform a rigid body transform across the 38 frames (block 303) to create a motion corrected dynamic PET volume, wherein motion is eliminated (block 304). In some embodiments, the PET processing engine may utilize the FMRIB software library (FSL) to conduct the aforementioned motion correction process.

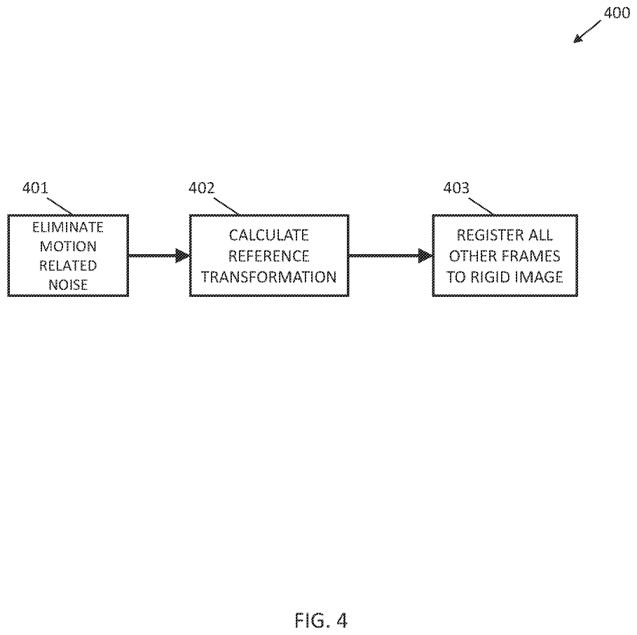

Next, a new average frame of all the motion corrected PET frames was resliced and co-registered into MRI space using the T1 weighted MRI (see blocks 401-403 of process 400 in FIG. 4) using non-rigid transforms to generate a transformation matrix. This was used, in turn, to generate a co-registered dynamic PET volume. For example, blocks 401-403 of FIG. 4 illustrate exemplary steps for dynamic PET MR co-registration conducted by a PET processing engine. In some embodiments, co-registration of the dynamic PET volume with high resolution MRI may be performed using FSL's linear registration tool, FLIRT, using a non-rigid transform. The average motion corrected dynamic PET volume was resliced and co-registered with MRI to generate a transformation matrix using non-rigid transforms, which was in turn utilized to generate a co-registered dynamic PET volume.

### MCIF and Parametric FDG PET Quantification

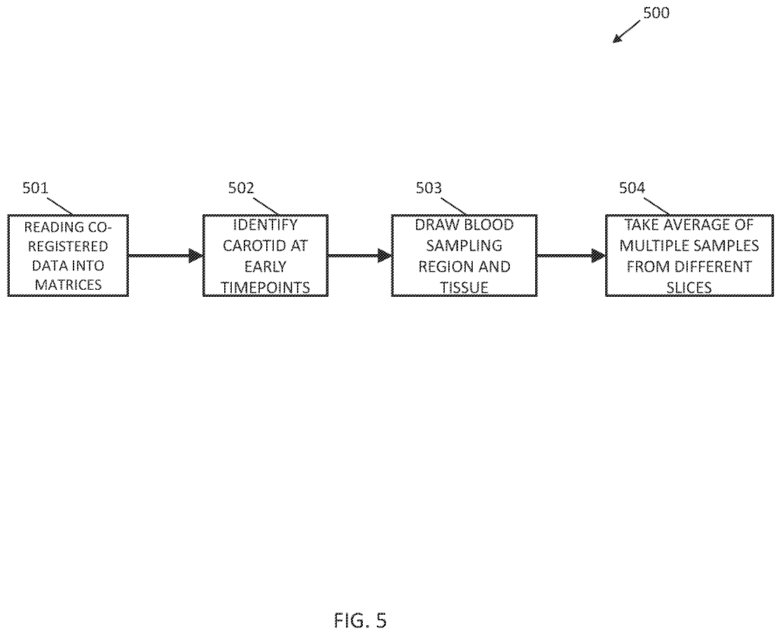

In some embodiments, co-registered volumes may then be used by the PET processing engine (e.g., PET processing engine 208 in FIG. 2) in the creation of objective parametric PET maps from a model corrected blood input function (MCIF) corrected for partial volume (PV) averaging and spill-over (SP) contamination. This process started with PET processing engine 208 obtaining an image-derived blood input function (IDIF), which can be taken from the internal carotid artery location after being identified at an early time frame for each patient. This process can be repeated twice on each artery for a cohort average of 4 ROIs of the left and right internal carotid arteries per subject (see blocks 501-504 or process 500 in FIG. 5). These ROIs were applied by PET processing engine 208 to all the motion-corrected 38 PET frames to generate an average model of the four blood time activity curves (PET_{IDIF}). This model IDIF, while correcting the blood input for PV and tissue SP contamination, can be written as:

$${Model}_{IDIF , i} = \frac{{\int }_{{t}_{b}^{i}}^{{t}_{e}^{i}} [ {S}_{Tb} {C}_{T} ( t ) + {r}_{b} {C}_{a} ( t ) ] dt}{{t}_{e}^{i} - {t}_{b}^{i}} , \qquad ( 1 )$$

in which S_{Tb}=spillover (SP) contamination from the tissue to the blood at late time points, r_{b}=blood recovery coefficient, and t_{b} and tₑ=beginning and end of a time frame, respectively. In some embodiments, C_{T}(t), the model tissue, was obtained by PET processing engine 208 solving the FDG transport differential equations from blood to tissue spaces and where Cₐ(t) is 7-parameter model blood for FDG transport. The above model IDIF may then be optimized using the following two objective functions:

$${O}_{1} ( p ) = {\sum }_{i = 1}^{n} {( {Model}_{IDIF , i} - {PET}_{IDIF , i} )}^{2} \qquad ( 2 )$$

$${O}_{2} ( p ) = {( {ModelPeak}_{IDIF} - {PETPeak}_{IDIF} )}^{2} \qquad ( 3 )$$

$$O ( p ) = {O}_{1} ( p ) + {O}_{2} ( p ) \qquad ( 4 )$$

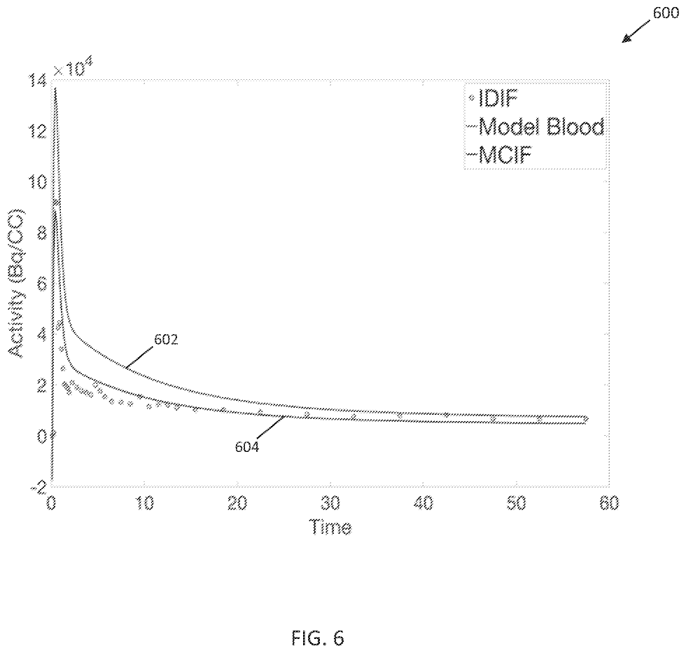

Notably, ModelPeak was computed by PET processing engine 208 from the model equations for the IDIF (Model_{DIF}) (equation 1). PETPeak values were derived by PET processing engine 208 from the dynamic PET blood images for each patient. Optimization of O(p) can be accomplished by using MATLAB's non-linear regression analysis toolkit and “fmincon” function, yielding the estimated MCIF for each subject (see plot line 604 in graph 600 of FIG. 6). In particular, FIG. 6 shows a graph 600 depicting an MCIF that was computed by optimizing two cost functions (see equations 2 and 3 above). The first cost function, O₁(p), minimizes the square of the difference between Model Blood and IDIF across the entire dynamic range. The second cost function, O₂(p), minimizes the square of the difference between Model Blood and IDIF peak values. The net cost function, O(p), is shown as a solid line 604 that is fit to the IDIF shown in circles. The solid line 602 represents the computed MCIF corrected for partial volume effects at the early time points and spill over contamination at the late time points.

Each voxel of the dynamic co-registered PET data was then independently fed (e.g., by PET processing engine) into a 4 parameter 3-compartment kinetic model, along with the computed MCIF to compute (by the PET processing engine) whole brain parametric (K₁, k₂, k₃, Kᵢ and TBV) maps using the following equation:

$${C}_{m} ( t ) = \frac{1}{( {t}_{2} - {t}_{1} )} \text{} {\int }_{{t}_{1}}^{{t}_{2}} \{ ( 1 - TBV ) [ \text{} \frac{{K}_{1} {k}_{3}}{{k}_{2} + {k}_{3}} {\int }_{0}^{T} {C}_{a} ( u ) du + \frac{{K}_{1} {k}_{2}}{{k}_{2} + {k}_{3}} {\int }_{0}^{T} {C}_{a} ( u ) \cdot {e}^{- ( {k}_{2} + {k}_{3} ) ( T - u )} du ] + TBV \cdot {C}_{a} ( T ) \} dT$$

where, Cₐ(t) is the computed MCIF and K₁-k₃ are the kinetic parameters. Further, TBV is the total blood volume accounting for the spill-over contamination from the blood to the tissue at the early time points and (1-TBV) accounts for the partial volume averaging for the tissue voxel. Cₘ(t) is the measured tissue voxel time activity curve. One subject's co-registered data (512 pixels×512 pixels×111 slices×38 frames) on average contained over one billion voxels, each with its own set of equations to solve.

To accomplish each of these computations (e.g., by PET processing engine 208) in a reasonable time frame, parallelization of multicore high-performance computers using the Rivanna HPC cluster at UVA may be used to compute whole-brain parametric maps for all patients. In some embodiments, the underlying software of PET processing engine 208 can be configured to interact with a pool of specialized computation hardware to build and tune parameters for a network of computation nodes to accomplish the task. Briefly, specialized command nodes are given instructions on how to recruit computation nodes, allocate resources, and divide up the task so that each voxel can be computed independently without repeating any computation. The command node then reassembles all of the voxel data into a final output matrix of parameters. It then instructs a smaller number of nodes to assemble and write maps for each of the parameters, which are returned alongside the final parameter matrix. In the effort to achieve the eventual goal for the process to output relatively quick predictions for regions of interest using an automated version of the method processing pipeline, the parallelization was optimized to its current runtime of approximately 2.5 hours for one subject's full brain image. The process was completed this way in order to collect a complete data set for further research. The time frame can be further reduced to fractions of an hour if the segmentation of abnormality areas in each subject's data is accomplished first. As such, drastically fewer equations are solved by PET processing engine 208 to collect parameter data for the abnormal tissue.

### Segmentation

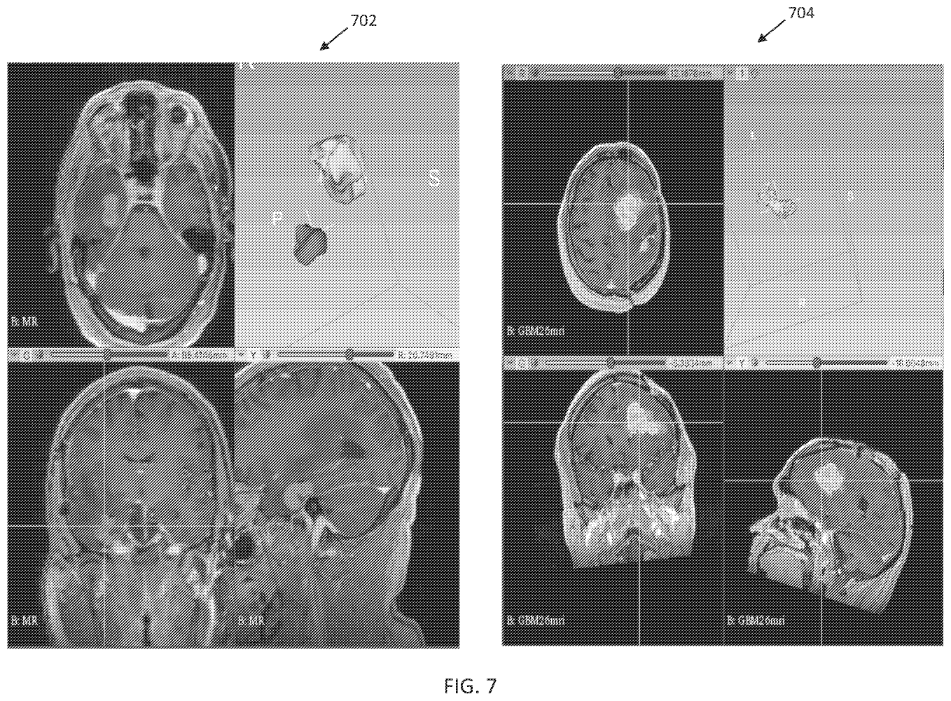

In some embodiments, 3D masks of abnormality volumes generated from the T1-weighted MR images may be semi-automatically segmented using a segmentation engine for each subject. In some embodiments, segmentation engine 230 includes and/or controls a 3D slicer image computing platform (or “3D Slicer”). For example, each subject may have between 1 and 5 abnormality areas of interest. Notably, images 702-704 of FIG. 7 illustratesan example tumor masking process using segmentation engine 230 (e.g., 3D Slicer). In particular, segmentation engine 230 may utilizes a rough/approximate outline of the tumor areas (or other abnormality volumes) across several slices (e.g., individual ‘seeds’ or seed images), which may then be expanded automatically using the segmentation engine and/or 3D Slicer to sample the entire tumor volume.

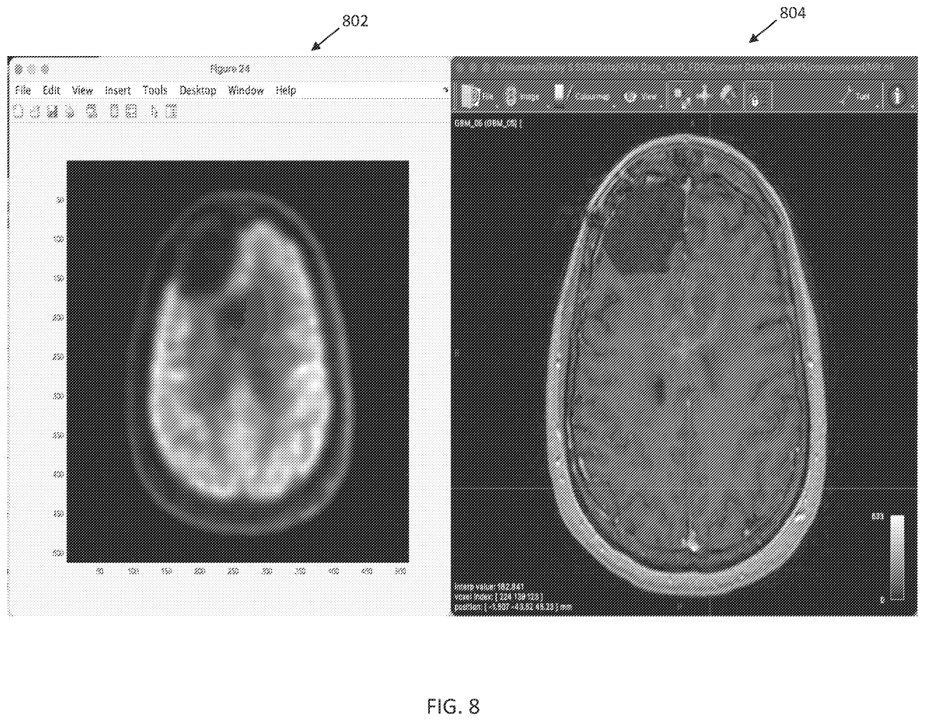

In addition to drawing the approximate outline of the abnormality area in a few slices, several example area masks (or slice masks) are drawn and entered into the program as “seeds” or “seed images” by segmentation engine 230. Using pre-trained processes, segmentation engine 230 and/or the 3D slicer then expands these seed images automatically to collect the entire abnormality area. In some embodiments, Smoothing is then semi-automatically performed by the segmentation engine 230 to get both a more conservative and realistic area mask of the abnormality volume. Area masks may then be verified by clinical experts and exported. In some embodiments, these area masks may be overlaid and/or dropped on to the co-registered parametric PET maps by segmentation engine 230 (e.g., using MATLAB software) to generate average kinetic rate parameters or constants (e.g., K₁-k₃ and Kᵢ) and total blood volume (TBV) for each abnormality volume across all subjects (e.g., see FIG. 8). Notably, example images 802 and 804 of FIG. 8 illustrate tumor masks (e.g., area masks or slice masks) generated from MR images overlaid and/or dropped onto parametric PET maps. The area masks generated by segmentation engine 230 (e.g., from 3D slicer on MR images) are exported and dropped onto to the compartment model computed parametric PET maps to generate kinetic rate parameters, such as average kinetic rate constants, K₁-k₃, Kᵢ and total blood volume, TBV.

### Classification

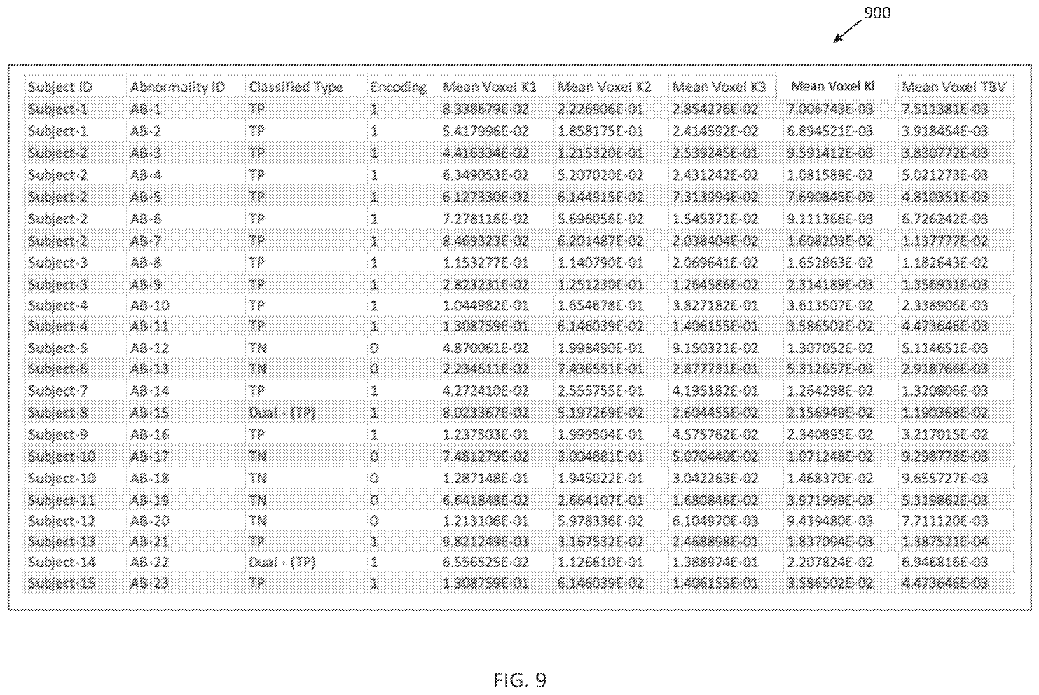

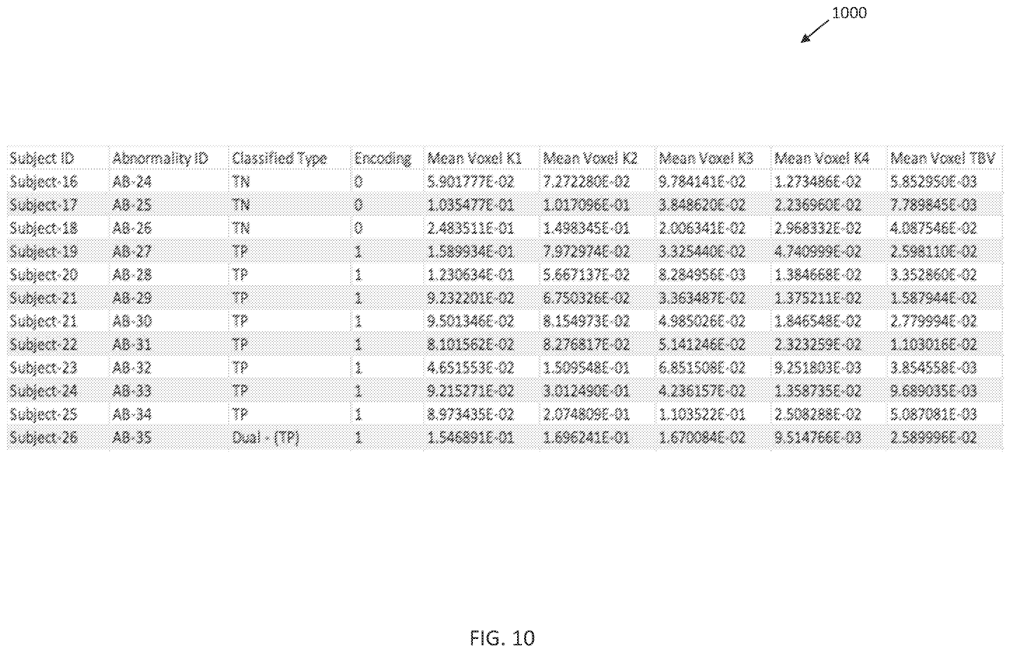

In some embodiments, PET processing engine 208 in FIG. 2 includes a classification manager 232 that is configured to perform the following classification tasks. For example, the above kinetic rate constants were collected and imported into R statistical programming language for analysis by the classification manager. In order to show the usefulness of this data, prediction results were analyzed using two classification methods (e.g., logistic and ridge linear regression) to get the more accurate predictions. Data from the abnormality regions (e.g., 35 regions) may randomly split into three groups for prediction model building: a training set to construct the foundation, a validation set for fine tuning certain methods, and a testing set to evaluate the model's performance. In some embodiments, The training data included 23 abnormalities (see Table 900 in FIG. 9), while the validation and testing sets include the remaining 12 abnormalities to be split as needed for ridge regression (see Table 1000 in FIG. 10). In particular, table 900 illustrates an example training data set with assignment of 1 for TP and 0 as TN to abnormalities based on clinical outcomes which was a combination of imaging follow-up (e.g., MRI) and surgical pathology. Table 1000 depicts an example validation and testing set with assignment of 1 for TP and 0 as TN based on clinical outcomes (i.e., target site condition assessment), which was a combination of imaging follow-up (e.g., MRI) and surgical pathology.

In some embodiments, the first exploratory technique includes constructing a logistic regression model (e.g., logistic regression engine) based on clinical outcomes. The clinical outcomes (e.g., target site condition assessments) indicated either tumor progression (TPR) or treatment related necrosis (TRN) for any given patient subject. In some embodiments, the kinetic rate parameters were first labeled by the classification manager 232 for training the logistic regression model with 17 TPR instances assigned as 1 (i.e., assigned condition; ground truth label y) and 6 instances of TRN assigned as 0 (i.e., assigned condition; (1-y)) across the pool of 23 training abnormalities. A logit function (L) of the form,

$$L = {b}_{0} + {b}_{1} {x}_{1} + {b}_{2} {x}_{2} + {b}_{3} {x}_{3} + {b}_{4} {x}_{4} + {b}_{5} {x}_{5} \qquad ( 6 )$$

was defined, where b₀ is the intercept and b₁-b₅ are slope parameters for the kinetic rate constants, K₁ (x₁), K₂(X₂), k₃(x₃) and net influx constant Kᵢ (x₄) and total blood volume, TBV(x₅). Then exponential of the logit function, L, (exp(L)) was computed followed by computing the probability,

$$\hat{y} = {e}^{L} / ( 1 + {e}^{L} ) \qquad ( 7 )$$

and the log likelihood (LL) as follows,

$$LL = {\sum }_{i = 1}^{n} {y}_{i} ln ( \hat{y} ) + ( 1 - {y}_{i} ) ln ( 1 - \hat{y} ) \qquad ( 8 )$$

Equation 8 can be maximized by the classification manager using a gradient ascent algorithm by adjusting the parameters b₀-b₅. The logistic model with the optimized parameters were then tested by using data from the validation and test data set and evaluated using Wald z-statistics.

Next, ridge regression can be performed (Equation 9) by adding a weighted L2 regularization parameter, λ, to the loss function and a (23,6,6) split of abnormalities into training, validation, and test sets, respectively. The ridge loss function was defined as:

$${L}_{ridge} ( \hat{β} ) = {\sum }_{i = 1}^{n} {( {y}_{i} - {x}_{i} \hat{β} )}^{2} + λ {\sum }_{j = 1}^{m} {\hat{β}}_{j}^{2} \qquad ( 9 )$$

where \hat{β} represents the slope parameters analogous to b's defined above. In order to determine the proper λ for regularization, regular linear regression models were built for a large number of configurations for abnormality assignments to each of the three groups. From there, predictions were made and the model was evaluated in the same manner as the logistic regression model. All of the above analysis techniques were assisted by R statistical computing language.

### Results

In some embodiments, the computed PV recovery for the blood input on an average across 26 patients was approximately 88% and the average SP contamination from the tissue to the blood was approximately 11%. The optimized logistic parameters b₀-b₅ were computed to be 2.905 (intercept), −29.641 (slope for K₁), −8.151 (slope for k₂), 4.182 (slope for k₃), 28.667 (slope for Kᵢ) and 112.572 (slope for TBV) respectively. Using cross validation, the optimal A for ridge was determined to be 0.398. The optimized ridge parameters were computed to be 0.783 (intercept), −0.753 (K₁), −0.853 (k₂), 0.349 (k₃), 3.601 (Kᵢ), and 2.547 (TBV) respectively. Combined, the training and testing process of parametric PET data sets of 23 abnormalities across 15 subjects and for 12 test abnormalities across 11 subjects was repeated several times each with its own unique random configuration of testing and training data. The logistic regression model was used to classify TRN or TPR with a 0.6 decision boundary, meaning that any predictions with a value 0.6 or over were considered to be a prediction for TPR, while lower values were considered to be a prediction of TRN. Logistic regression predicted in aggregate across all random configuration iterations with approximately 83% accuracy.

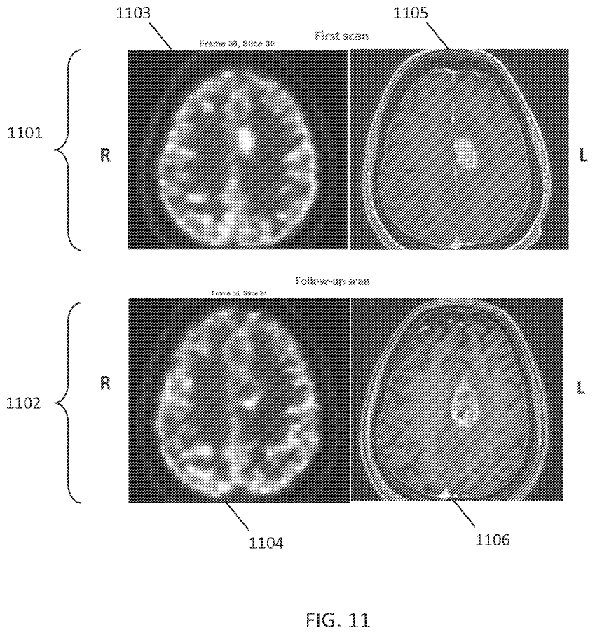

Case study: Only one out of N=26 subjects underwent a follow-up dynamic PET study. Scan 1101 of FIG. 11 shows the last time frame of the dynamic PET study and T1-weighted contrast enhanced MR image of the same subject. The diagnosis on static PET, despite high PET uptake, TRN was favored over TPR due to MR appearance (scan 1101 in FIG. 11). MR imaging follow up and surgical pathology (ground truth) indicated TRN. This subject was monitored with a 3-month follow up MR and dynamic PET study (scan 1102 in FIG. 11). The diagnosis on static PET indicated TRN with residual TPR along the margins. MR imaging follow up and surgical path indicated TPR. The disclosed classification scheme which was trained as a TRN based on MR imaging follow up and surgical pathology (scan 1101 in FIG. 11) was predicted to be a TPR based on a probability score of 0.665 and decision boundary of 0.6. Logistic regression clearly indicated a TPR on the follow up study (scan 1102 in FIG. 11) with a probability score of 0.861, which agreed with surgical pathology.

Notably, scan 1101 in FIG. 11 depicts the last time frame of the dynamic PET study (image 1105) and T1-weighted contrast enhanced MR image (image 1103) of the same subject. This subject was monitored with a 3-month follow up MR and dynamic PET study (see scan 1102). Logistic regression model trained as a TRN based on MR imaging follow up and surgical pathology was predicted to be a TPR based on a probability score of 0.665 and decision boundary of 0.6 for the first scan. Logistic regression clearly indicated a TPR for the follow up scan 1102 with a probability score of 0.861

The ridge regression model was used to classify TRN or TPR with a lower 0.5 decision boundary, which is a bit more intuitive as it accounts for scaled data. The ridge regression model predicted in aggregate across all configurations with approximately 84% accuracy. Notably, the Wald z-statistics for K₁, and k₂ were −1.724 and −1.942 respectively in logistic regression. Considering the reduced magnitude through coefficient regulation in ridge regression, these parameters would appear to be the most impactful and share multicollinearity with the Kᵢ and TBV parameters.

### DISCUSSION

MRI is the standard of care for clinical imaging to evaluate for tumor progression following treatment. However, due to the overlapping appearance of tumor progression (TPR) and brain tissue changes induced by chemoradiotherapy (“treatment effect” or treatment related necrosis (TRN)) on contrast enhanced MRI, sensitivity and specificity of MRI are not adequate in many cases to direct patient management. Advanced MR techniques also frequently render overlapping metrics between tumor progression and treatment effect. Notably, ¹⁸F-FDG PET is in principle an excellent technique to achieve this differentiation as there is increased glucose metabolism in malignant cells, including glioma and metastases, compared with benign or normal tissues. Also, changes in cell metabolism may precede anatomic changes when a tumor responds to therapy, which could be detected by FDG PET. The standard of care for interpreting FDG PET is qualitative and visual by performing side-to-side comparisons and by comparing to the other regions in the cortex. Standardized uptake values (SUV) widely used as a semi-quantitative measure for tumors outside the brain has proven to be unreliable for this indication, as it can depend on several factors such as body weight and blood glucose level. Several other methods have been proposed to overcome the challenge with SUV measurements in the evaluation of brain gliomas with FDG, however none of these has been proven to be reliable.

Amino acid tracers including 3,4-dihydroxy-6-[¹⁸F] fluoro-L-phenylalanine (18F-FDOPA), 11C-Methionine (11C-MET) and 18F-fluoroethyl-L-tyrosine (18F-FET) have emerged as alternative tracers to FDG, as it is transported by the L-amino acid transporters which are overexpressed in most gliomas. The diagnostic accuracy of dynamic 18F-FDOPA PET has been evaluated in imaging brain tumors in comparison to static FDG using standard of care SUV analysis. Recent work by Wardak et al. in a dynamic PET study indicated the combined use of 3′-deoxy-3′-18F-fluorothymidine (FLT), a tracer to measure cell proliferation, and FDOPA data in predicting the overall survival of patients with recurrent brain tumors using multiple linear regression analysis. In another study, the performance of 18F-FET was compared to FDG for the diagnosis and grading of brain tumors. It was concluded that FET-PET performed better than FDG especially in assessing a new isolated brain tumor. In a recent meta-analysis, FET, MET (both approved for use only in Europe), FDOPA, and FLT PET were suggested to have a higher diagnostic accuracy than standard of care MRI in the differentiation of actual tumor progression from treatment-related changes. In another recent study, however, it was concluded the diagnostic accuracy of FET PET, to differentiate these entities, may be slightly inferior compared to previously reported studies using the same tracer. Although most studies indicate that the studied radiotracers offer benefits over the widely used radiotracer (FDG), none of them appear to have a clear advantage over the others in all aspects of brain tumor imaging. In addition, none of these studies utilized the combined utility of advanced kinetic PET analysis and supervised machine learning in the differential diagnosis of TPR vs TRN using dynamic FDG PET. Preliminary results derived from highly quantitative model blood input with PV and SP corrections and peak fitting cost function, accurate brain tumor segmentations and supervised machine learning including logistic and linear ridge regression models with proper regularization predicted TPR vs TRN at the level of 83% and 84% accuracy respectively.

In conclusion, PV recovery and SP contamination resulted in accurate computation of the blood input function non-invasively from dynamic FDG PET images. Simple logistics and linear regression models produced in R were successful in distinguishing between TRN and TPR at a promising level considering the limited amount of training data available and show great potential for a clinical tool with further studies. Based on the evaluation metrics and Wald z-statistics, the optimized parameters from logistic regression analysis appear largely influenced by the first and second rate constants. This suggests that the parameters K₁, and k₂ may be particularly relevant predictors for classifying tumor progression versus treatment related necrosis in any given abnormality. Based on these initial findings, it is predicted that automated derivation of the blood input and brain tumor segmentations and more thorough statistical analysis with categorical variables and additional kinetic information at the voxel level using supervised machine learning can yield a superior prediction model. Unsupervised machine learning techniques could also be used to recognize patterns and important parameters. These findings can be used in pilot studies in evaluation of regression mapping in combination with dynamic FDG PET to predict tumor remission.

Differential diagnosis of tumor recurrence versus treatment effect (i.e. radiation necrosis or pseudo progression) in high grade glioma (HGG) and brain metastases patients is a significant clinical problem after patients undergo radiation therapy and chemotherapy. This aim will utilize advanced parametric PET analysis methods developed to perform improved differentiation of these entities thereby improving clinical outcomes and quality of life. Preliminary data can be obtained using a non-TOF and TOF PET scanner. There is compelling preliminary evidence that kinetic and wavelet transform analysis, when applied to dynamic data obtained using the non-TOF PET imager, is able to differentiate tumor recurrence from radiation effect. However, using the TOF PET imager, due to improved sensitivity, it has recently been determined that an additional tumor feature criterion (e.g., d8 member of the decomposition) may differentiate and also predict the above two entities.

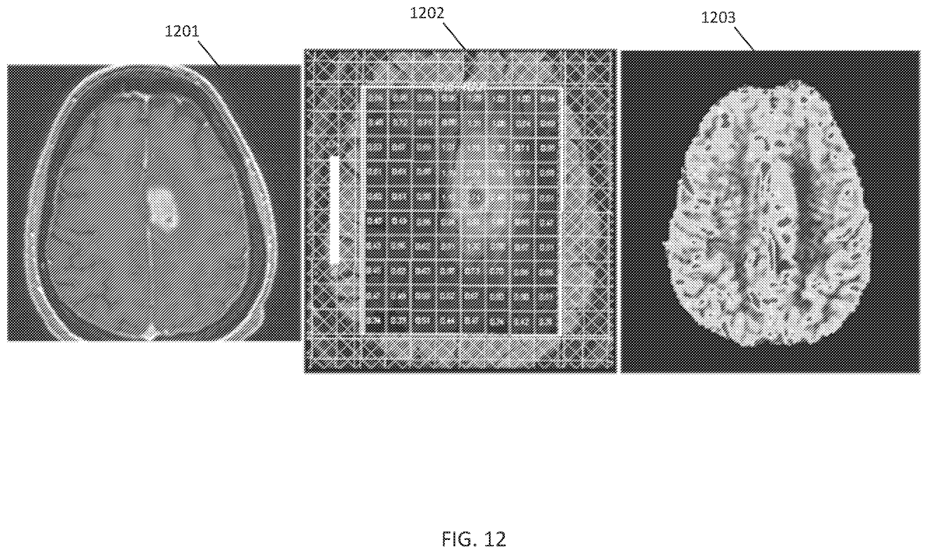

In FIG. 12, MR images including T1 weighted contrast enhanced (see image 1201), Multivoxel MR spectroscopy (e.g., echo time of 270 milliseconds) (see image 1202) and Dynamic susceptibility contrast perfusion weighted (DSC-PWI) imaging (see image 1203). The T1 weighted images suggested enhancing lesion which either represented tumor progression or treatment effect. The maximum choline/NAA ratio from the spectroscopy images and relative cerebral blood volume (rCBV) from the DSC-PWI images were computed to be 5.74 and 5.1 respectively, thus suggesting tumor progression. Returning to FIG. 1, images 101-102 show a dynamic PET images and plots 103-104 depict wavelet transform (WT) analysis. The first FDG PET scan post treatment with PARP inhibitor and radiation therapy indicated active tumor visually in PET images (last time frame of the dynamic data). However WT analysis (ROI 1) predicted radiation effect (plot 106), which agreed with the follow up scan (e.g., images 102). WT analysis on the residual tumor (ROI 2) further predicts radiation effect (plot 104), confirmed by 3-6 month interval multidisciplinary clinical evaluation.

In summary, there is preliminary evidence that dynamic PET imaging can assist with the differentiation of neoplasm recurrence from treatment-induced brain tissue changes. Addition of a FDA approved highly specific amino acid tracer, FDOPA, may enhance the analytical capabilities of parametric imaging with the TOF PET imager.

As indicated above, the current standard of care of evaluating PET data is highly qualitative and visual by performing side-to-side comparisons and by comparing to the other regions in the cortex. Standardized uptake values (SUV) has been widely used as a semi-quantitative measure in quantifying 18F-FDG brain PET data. However, SUV measurement has proven to be unreliable due to its dependence on body weight, injected dose and blood glucose levels. Recent development in quantification has involved collecting time-resolved 18F-FDG PET images (dynamic PET data), which may aid in differentiating necrotic from the recurrent tumor based on glucose uptake rates (Kᵢ) computations. However, computing Kᵢ in a 2-tissue compartment model is challenging due to the limited intrinsic spatial resolution of the state-of-art Siemens mCT PET scanner resulting in incomplete radioactivity recovery or partial volume (PV) averaging and hence spill-over (SP) of radioactivity from the surrounding regions into the regions of interest in a dynamic brain PET scan.

As such, the disclosed subject matter pertains to methods for improving the above differentiation by exploiting the diagnostic power of full dynamic/kinetic analysis primarily of the uptake data (and/or washout data). It is hypothesized that the improved methods adapted from other studies (e.g., mouse studies) in combination with wavelet transforms applied to early temporal variation in tissue time activity curves obtained from dynamic brain PET data in humans can provide functional information to classify viable or recurrent tumors from treatment-induced radiation necrosis. Preliminary results with F18-FDG using the conventional clinical whole body Siemens Biograph PET/C_{T}(mCT) scanner are very encouraging in the differentiation of post-treatment changes from recurrent cancer in GBM patients.

### Methods for Kinetic and Wavelet Analysis

In some embodiments, the dynamic FDG PET scan of the brain of a plurality of subjects (e.g., 8 patients ages 18 and above) with suspected tumor recurrence was performed using the Siemens Biograph mCT (PET/C_{T}) scanner, wherein each patient was first placed with the brain in the center of the field of view and a 30 minute scan was initiated in a list-mode format, followed immediately by ˜10 mCi of FDG injection intravenously over a ˜10-20 second period. A 10 minute static image was also acquired immediately after the dynamic scan. An MR scan of the same patient was performed a day prior to the dynamic PET scan.

### Kinetic Modeling

In some embodiments, the PET processing engine 208 (as shown in FIG. 2) can be configured to conduct kinetic and wavelet analysis of dynamic FDG PET data in human GBT. For example, PET images can be reconstructed by the PET processing engine, with attenuation correction, using an OSEM iterative algorithm with 24 OSEM subsets and 2 iterations into the following dynamic frames with time in seconds: 12,10; 8,30; 8,60; 2,180; 2,300. A Matlab software routine (e.g., included or managed by PET processing engine 208) can be used to draw regions of interest (ROI) on one of the carotid arteries and in the suspicious brain region (i.e., target site), to generate a blood time activity curve (TAC) and tissue TAC, respectively. A 3-compartmental tracer kinetic model with spill over (SP) and partial volume (PV) corrections can be applied (e.g., by PET processing engine 208) to compute either the parametric image (pixel-by-pixel) or regional value of FDG uptake rate, Ki (ml/min/g). Referring to the structural image from MRI and static PET scan image, the relationship of Ki in the regions of tumor, contralateral normal white matter and gray matter may be compared to determine the abnormality in the rate of FDG uptake. Based on a preliminary criterion of Ki_ratio=Ki (tumor): Ki (contralateral white matter), the tumors were classified as recurrent (Ki_ratio >1.5) or necrotic (Ki_ratio≤1.5).

### Wavelet Transform

Furthermore, the features embodied in the early temporal variation of the tissue TAC were also extracted using the Wavelet Transform (WT) algorithm (e.g., by PET processing engine 208 and/or WT analyzer 234) and used in tumor discrimination. Briefly, the TAC data was treated as a time-varying noise contaminated signal and decomposed by a ‘sym’ wavelet to level 8 in Matlab. At the d6 member of decomposition, the amplitude (positive peak value>=50), the number of peaks (>=2) of the wave and the lasting time (no peak after 4 minutes) were used as characteristics to discriminate the tumor from the normal tissue. In addition, at the d8 member of decomposition, only the amplitude (positive peak value>=50) was used (e.g., by PET processing engine 208 and/or WT analyzer 234) to discriminate the two entities. Thus, a tumor feature criterion may be proposed based on the above three values on d6 member together with one value of the d8 member of the wavelet transformation applied to tissue TAC.

### Results

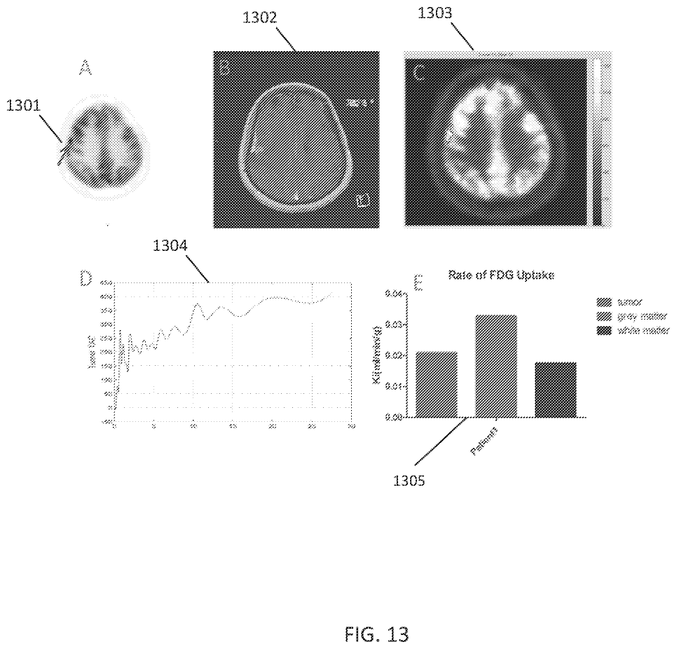

FIG. 13 shows representative MR and static and dynamic PET images of a patient subject with a brain tumor. The tissue TAC and the computed rate of FDG uptake, Ki, are also shown.

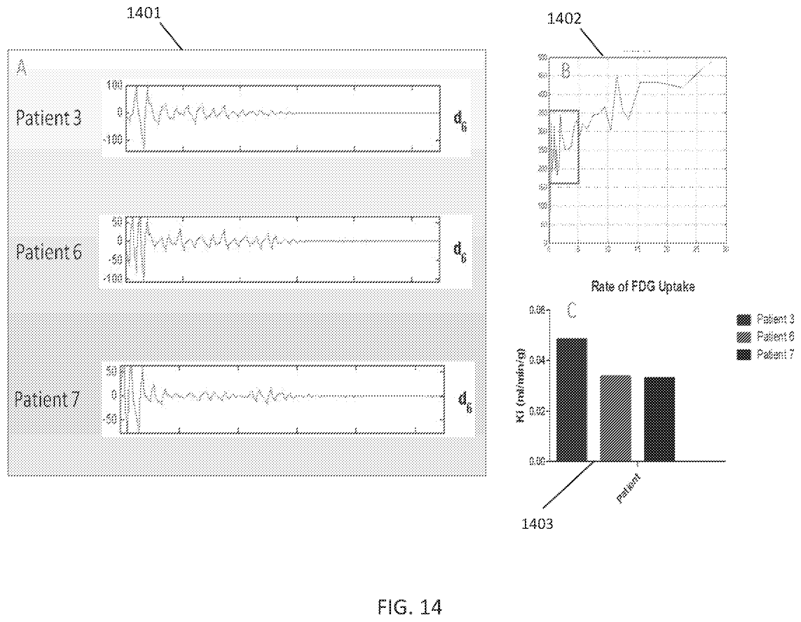

The consistency of the result from WT and FDG uptake rate analysis is illustrated in FIG. 14. The TAC signal as shown in graph 1402 was decomposed by the WT to extract the early temporal waves and then generate the d6 member of the signal (plots 1401). From the representative data based on 3 patients, it can be seen that higher d6 signal amplitude corresponds to higher FDG uptake rate (bar graph 1403).

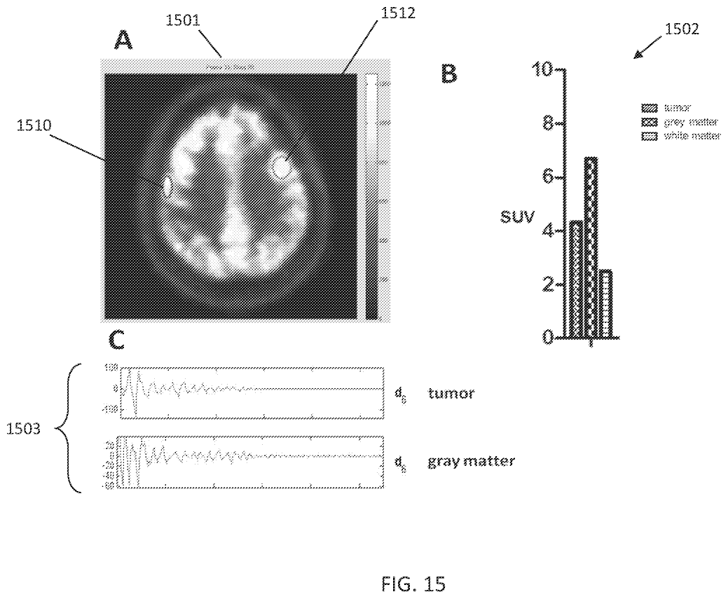

Furthermore, FIG. 15 illustrates that wavelet transform applied to the collected dynamic data separates the tumor from the contralateral gray matter, which is not possible from standard of care visual analysis and side-to-side SUV comparisons on a static FDG PET image. Notably, FIG. 15 illustrates a various representations pertaining to WT versus SUV analysis. For example, image 1501 depicts Regions of activity drawn on a dynamic PET image on the tumor (ROI area 1510) and contralateral grey matter (ROI area 1512). The tumor was identified using an MRI image (e.g., see MRI 1301 in FIG. 13). Bar graph 1502 depicts Standardized Uptake Values (SUV), and plot 1503 shows WT applied to the early temporal features of the tumor and gray matter TAC and the resulting d6 members of the decomposition. WT (e.g., via WT analyzer 234) differentiates tumor from gray matter, which is not possible from standard of care visual analysis and side-to-side SUV comparisons on static PET images.

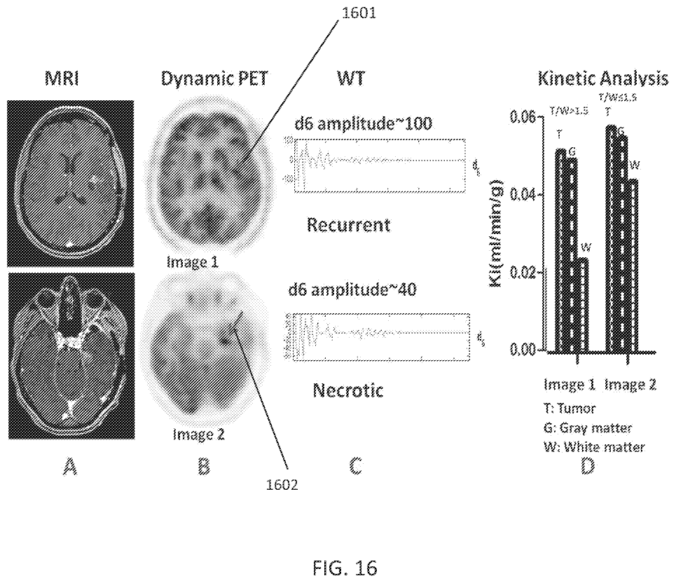

FIG. 16 shows that collective application of kinetic modeling and WT can differentiate a recurrent brain tumor from radiation necrosis, as identified from standard of care MRI. Notably, FIG. 16 depicts data pertaining to WT and kinetic analysis, such as (A) MR Image, (B) Dynamic PET, (C) WT analysis, and (D) Kinetic analysis. The arrow 1601 (and arrow 1602) points to the tumor. Kinetic analysis and WT differentiates recurrent tumor from radiation necrosis, which is not possible from standard of care SUV analysis using static PET images.

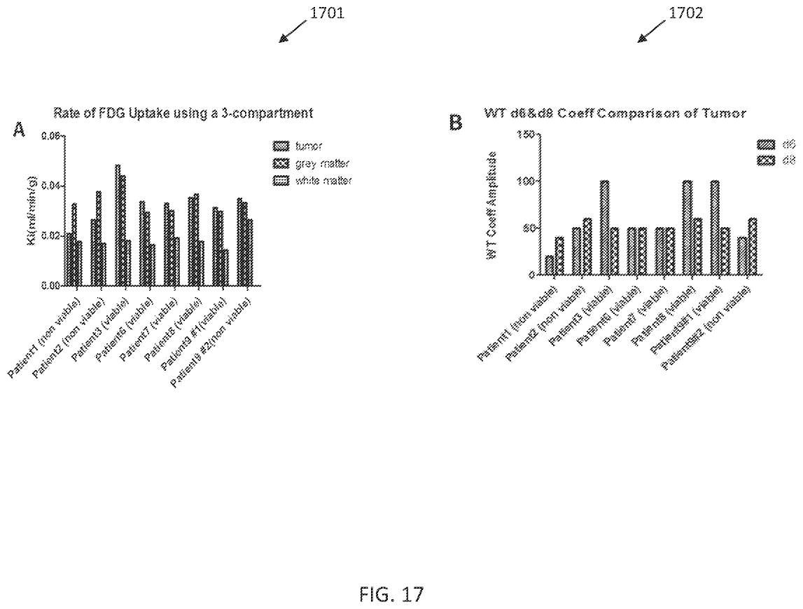

FIG. 17 shows a complete kinetic model and wavelet analysis of 8 patients including the d6 and d8 members of the decomposition. The results indicate that kinetic and WT analysis of dynamic FDG PET data matched with the diagnosis performed by an experienced physician based on MR images. Specifically, FIG. 17 illustrates the rate of FDG uptake and WT analysis in 8 patients imaged with the Siemens mCT PET/CT scanner. Graph 1701 shows the Ki analysis matched with the physician diagnosis based on MRI. Graph 1702 shows the WT analysis matched with the physician diagnosis based on MRI.

Evidence obtained with the standard PET/CT scanner shows that dynamic FDG imaging can assist with key answers to the efficacy of the therapy. The key issue is the differentiation of recurrence from treatment-related inflammation and scar tissue. FDG is taken up in both tissues and the conventional static imaging protocol does not separate the two tissues. Preliminary data indicate that kinetic model and WT applied to the collected dynamic data separates the two types of tissues by analyzing the uptake/washout curves using new powerful algorithms. It is expected that substantial increase in sensitivity using Time of Flight (TOF) dedicated brain PET imagers will greatly improve the accuracy of the analysis.

In some embodiments, the disclosed subject matter can be configured to conduct brain tumor differentiation using early temporal feature(s) of a time activity curve from dynamic FDG PET imaging. As indicated above, brain tumor detection and therapeutic evaluation are still challenging. Distinguishing between radiation necrosis and recurrent or viable residual tumor has proved to be a particularly difficult task. It is reported that nearly one third of the patients would have been treated inappropriately. The aim of the invention is to extract the time course features of the pharmacokinetics of the biomarker after venous injection using dynamic PET imaging and utilize the features as a new technique for brain tumor diagnosis other than MRI and static PET image. This method will provide functional information to classify tumor types, i.e. metastasis, recurrent tumor, or treatment induced changes such as radiation necrosis.

In some embodiments, a PET scan of a human brain was performed, wherein the patient subject with the brain in the center of field of view (CFOV) is first placed and a 30 minute scan initiated in list-mode format followed by ˜10 mCi of FDG injection intravenously (IV) over a 20-30 second period. A static 10 minute image was also acquired immediately after the dynamic scan. The PET images were reconstructed using OSEM iterative algorithm with 24 OSEM subsets and 2 iterations into the following dynamic frames: frames, time in seconds: 12,10;8,30;8,60;1,60. A software routine developed in Matlab was used to draw Region of Interest (ROI) on both the carotid artery and the interested brain tissue, to generate blood Time Activity Curve (TAC) and tissue TAC respectively.

A 3-compartmental tracer kinetic model with Spill over (SP) and Partial Volume (PV) corrections were applied to compute either the parametric image or regional value of FDG uptake rate Ki.

Referring to the structural image from MRI and static PET scan image, the relationship of Ki in region of tumor, contralateral normal white matter and gray matter were compared to determine the abnormity (e.g., abnormality volume) in the rate of FDG uptake. The metabolic activity of each lesion was characterized as hypometabolic, isometabolic, or hypermetabolic relative to normal contralateral white matter.

Furthermore, the features embodied in the early temporal variation of the tissue TAC were also extracted with the Wavelet Transform (WT) algorithm (e.g., WT analyzer 234 shown in FIG. 2) and applied for tumor discrimination. The TAC data was treated as a time-varying noise contaminated signal and decomposed by a ‘sym’ wavelet to level 8. At the d6 member of decomposition, the amplitude, the number of peaks of the wave and the lasting time were used as characteristics to discriminate the tumor from the normal tissue. A tumor feature criterion was proposed based on the above three values on d6 member of the wavelet transformation on TAC.

The combination of features from MRI, static PET image, FDG uptake of dynamic PET images and also temporal d6 decomposition signal of dynamic PET images were assimilated and utilized to improve the accuracy of tumor classification.

The current clinical gold standard, MRI, provides superior structural detail but poor specificity in identifying viable tumors in brain treated with surgery, radiation, or chemotherapy. The CT imaging has been unable to reliably distinguish recurrent tumor from radiation necrosis neither. Similarly, the current standard of care is very qualitative and visual by performing side-to-side comparisons and by comparing to the other regions in the cortex or performing graphical Patlak analysis.

In contrast, the disclosed subject matter is configured to utilize the WT algorithm (e.g., WT analyzer 234) to extract the early temporal signal waveform features of the TAC from the dynamic PET imaging and identifying the tumor according to the established criterion on the d6 member of the WT decomposition. A parametric image is built together with the image segmentation according to tumor type. The FDG update rate of the segmented regions are also calculated by the WT engine (e.g., WT analyzer 234). The above two functional information from dynamic FDG PET can be combined with the structural imaging modality, i.e., MRI or CT for the final tumor diagnosis.

A representative application of the method is shown in FIG. 13. As mentioned previously, FIG. 13 illustrates the procedure of using static PET (image 1301) and MRI (image 1302) as clues to first locate the tumor ROI on the dynamic image (image 1303). The measured tumor TAC (plot 1304) and also the computed rate of FDG uptake (graph 1305) are also shown.

Returning to FIG. 14, this figure illustrates the consistency of the result from wavelet transform (WT) and FDG uptake rate analysis. The TAC signal as shown in graph 1402 was decomposed by the WT to extract the early temporal waves and then generate the d6 member of the signal (plots 1401). From the representative data shown in 3 patients, it can be seen that higher d6 signal amplitude has higher FDG uptake rate. Bar graph 1403 depicts the comparative rate of FDG uptake.

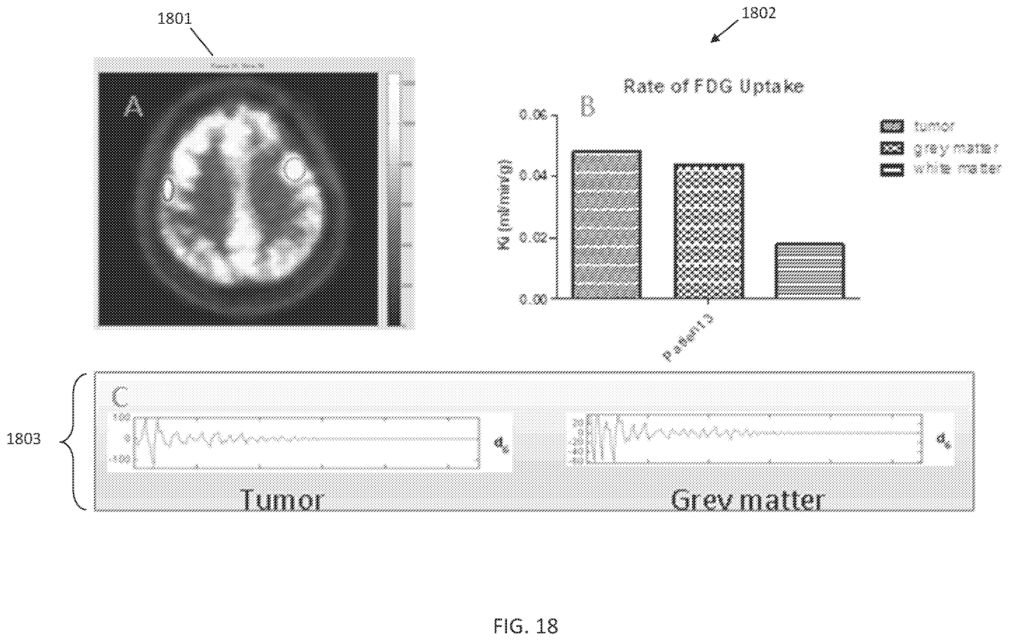

For example, scan 1801 of FIG. 18 illustrates a representative dynamic PET image depicting the tumor and grey matter have same FDG concentration. Likewise, bar graph 1802 illustrates the rate of FDG uptake showing that tumor has same level of uptake with grey matter. Notably, FIG. 18 indicates that although the FDG concentration (scan 1801) and uptake rate (graph 1802) are nearly the same between tumor and grey matter in the brain, the amplitude of the d6 member from the WT decomposition are significantly different (see plots 1803). In particular, plots 1803 indicate the amplitude of the d6 member of the WT decomposed signal corresponding to each of the tumor and grey matter, and showing that the tumor related amplitude has a significantly higher value.

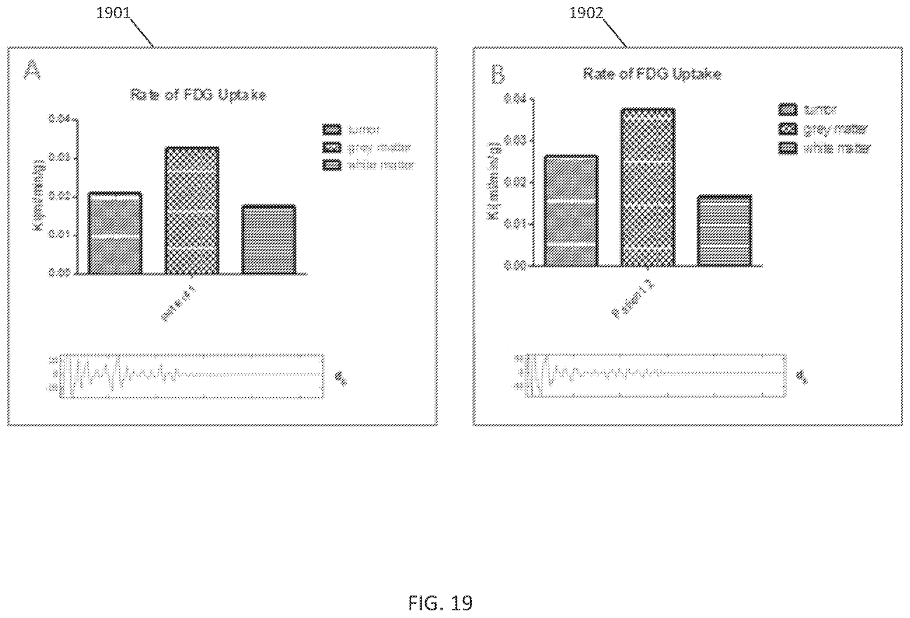

FIG. 19 shows that the amplitude of the d6 member from WT decomposition can be used to discriminate tumor progression. For example, the bar graph in section 1901 illustrates the rate of FDG uptake and d6 signal of pseudo-progression Glioblastoma tumor. Similarly, the bar graph in section 1902 illustrates the rate of FDG uptake and d6 signal of a low grade superficial tumor.

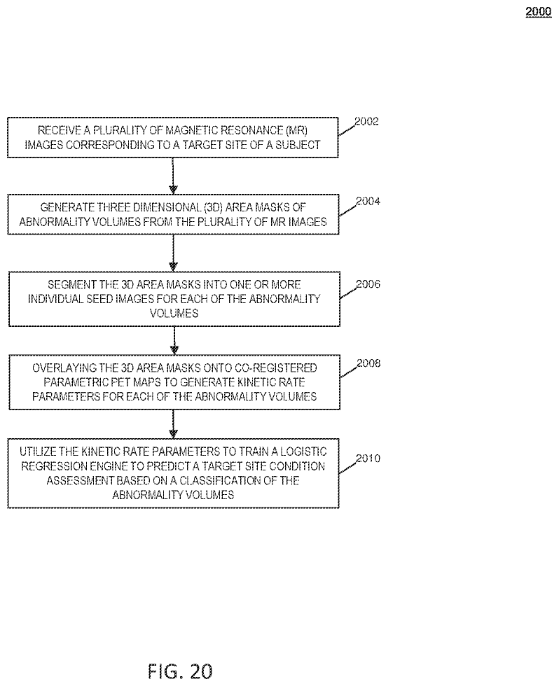

FIG. 20 is a flow chart illustrating an example process for performing FDG positron emission tomography (PET) quantification, segmentation, and classification of abnormalities according to an embodiment of the subject matter described herein. In some embodiments, method 2000 depicted in FIG. 20 is an algorithm, program, or script stored in memory that when executed by a processor performs the steps recited in steps 2002-2008. In some embodiments, method 2000 represents a list of steps embodied in the underlying software code programming or rules of the segmentation engine and/or classification manager in the host computing platform device shown in FIG. 2.

In step 2002, method 2000 includes receiving a plurality of MR images corresponding to a target site of a subject. In some embodiments, a PET processing engine and/or its segmentation engine is configured to receive MR images of a patient's brain. The PET processing engine may also be configured to receive parametric PET map scans associated with the same target site (i.e., the patient's brain).

In step 2004, method 2000 includes generating 3D area masks of abnormality volumes from the plurality of MR images. In some embodiments, the PET processing engine is configured to inspect the received MR images and detect abnormality volumes (e.g., brain tumor(s)) in the same. Upon detecting the presence of an abnormality volume, the PET processing engine is configured to generate a 3D area mask (or slice mask) of the inspected MR image.

In step 2006, method 2000 includes segmenting the 3D area masks into one or more individual seed images for each of the abnormality volumes. In some embodiments, the segmentation engine utilizes a three dimensional image slicing platform (e.g., 3D Slicer) to segment the generated 3D area masks into individual seed images (or “seeds”). In step 2008, method 2000 includes overlaying the one or more individual seed images onto co-registered parametric PET maps to generate kinetic rate parameters for each of the abnormality volumes. In some embodiments, the PET processing engine and/or segmentation engine is configured to overlay (or drop) the individual seed images created in step 2006 onto the co-registered parametric PET maps (of the same target site). By overlaying these two images, the PET processing engine is configured to generate kinetic rate parameters for each of the identified abnormality volumes (e.g., brain tumors).

In step 2010, method 2000 includes utilizing the kinetic rate parameters to train a logistic regression engine to predict a target site condition assessment based on a classification of the abnormality volumes. In some embodiments, the PET processing engine and/or classification manager is configured to use the generated kinetic rate parameters (e.g., kinetic rate constants) as training input for the logistic regression engine. Such a trained logistic regression engine can then be used to predict a target site condition assessment based on a classification of the abnormality volumes (e.g., determine whether a brain tumor is associated with tumor progression (TP) or tumor necrosis (TN)). In some embodiments, the logistic regression engine is subjected to supervised machine learning (ML) to classify the abnormality volumes.

It will be understood that various details of the presently disclosed subject matter may be changed without departing from the scope of the presently disclosed subject matter. Furthermore, the foregoing description is for the purpose of illustration only, and not for the purpose of limitation.

What is claimed is:

1. A method for performing FDG positron emission tomography (PET) quantification, segmentation, and classification of abnormalities, the method comprising:
receiving a plurality of magnetic resonance (MR) images corresponding to a target site of a subject;
generating three dimensional (3D) area masks of abnormality volumes from the plurality of MR images;
segmenting the 3D area masks into one or more individual seed images for each of the abnormality volumes;
overlaying the one or more individual seed images onto co-registered parametric PET maps to generate kinetic rate parameters for each of the abnormality volumes; and
utilizing the kinetic rate parameters to train a logistic regression engine to predict a target site condition assessment based on a classification of the abnormality volumes.

2. The method of claim 1 wherein the MR images are T1-weighted.

3. The method of claim 1 wherein overlaying the 3D area masks onto co-registered parametric PET maps generates a total blood volume (TBV) parameter for each of the abnormality volumes.

4. The method of claim 1 wherein the target site condition assessment includes a tumor progression (TPR) assessment or a treatment related necrosis (TRN) assessment.

5. The method of claim 1 wherein one or more wavelet transforms are utilized to determine the kinetic rate parameters.

6. The method of claim 1 wherein the logistic regression engine is subjected to supervised machine learning (ML) to classify the abnormality volumes.

7. The method of claim 1 comprising receiving the co-registered parametric PET maps corresponding to the target site of the subject.

8. A system for performing FDG positron emission tomography (PET) quantification, segmentation, and classification of abnormalities, the system comprising:
a PET scanner device configured for configured for collecting volumetric radioactive measurement data associated with an administered radioactive tracer present in a target site of a subject over multiple scanning intervals and generating associated parametric PET maps of the target site;
a magnetic resonance (MR) imaging scanner device configured for capturing a magnetic resonance image of the target site; and
a dynamic PET platform comprising:
at least one processor;
a memory element; and
a PET processing engine stored in the memory element and when executed by the at least one processor is configured for receiving a plurality of MR images corresponding to a target site of a subject, generating three dimensional (3D) area masks of abnormality volumes from the plurality of MR images, segmenting the 3D area masks into one or more individual seed images for each of the abnormality volumes, overlaying the one or more individual seed images onto co-registered parametric PET maps to generate kinetic rate parameters for each of the abnormality volumes, and utilizing the kinetic rate parameters to train a logistic regression engine to predict a target site condition assessment based on a classification of the abnormality volumes.

9. The system of claim 8 wherein the MR images are T1-weighted.

10. The system of claim 8 wherein the PET processing engine is configured to overlay the 3D area masks onto co-registered parametric PET maps to generate a total blood volume (TBV) parameter for each of the abnormality volumes.

11. The system of claim 8 wherein the target site condition assessment includes a tumor progression (TPR) assessment or a treatment related necrosis (TRN) assessment.

12. The system of claim 8 wherein one or more wavelet transforms are utilized to determine the kinetic rate parameters.

13. The system of claim 8 wherein the logistic regression engine is subjected to supervised machine learning (ML) to classify the abnormality volumes.

14. The system of claim 8 wherein the PET processing engine is configured to receive the co-registered parametric PET maps corresponding to the target site of the subject.

15. One or more non-transitory computer readable media having stored thereon executable instructions that when executed by a processor of a computer cause the computer to perform steps comprising:
corresponding to a target site of a subject;
generating three dimensional (3D) area masks of abnormality volumes from the plurality of MR images;
segmenting the 3D area masks into one or more individual seed images for each of the abnormality volumes;
overlaying the one or more individual seed images onto co-registered parametric positron emission tomography (PET) maps to generate kinetic rate parameters for each of the abnormality volumes; and
utilizing the kinetic rate parameters to train a logistic regression engine to predict a target site condition assessment based on a classification of the abnormality volumes.

16. The one or more non-transitory computer readable media of claim 15 wherein the MR images are T1-weighted.

17. The one or more non-transitory of claim 1 wherein overlaying the 3D area masks onto co-registered parametric PET maps generates a total blood volume (TBV) parameter for each of the abnormality volumes.

18. The one or more non-transitory computer readable media of claim 15 wherein the target site condition assessment includes a tumor progression (TPR) assessment or a treatment related necrosis (TRN) assessment.

19. The one or more non-transitory computer readable media of claim 15 wherein one or more wavelet transforms are utilized to determine the kinetic rate parameters.

20. The one or more non-transitory computer readable media of claim 15 wherein the logistic regression engine is subjected to supervised machine learning (ML) to classify the abnormality volumes.
